(19) United States
(12) Patent Application Publication
(10) Pub. No.: US 2006/0286136 A1
(43) Pub. Date: Dec. 21, 2006

(54) Surface treatment of biomedical implant for improved biomedical performance

(75) Inventors: Kevin J. Moeggenborg, Naperville, IL (US); Steven K. Grumbine, Aurora, IL (US); Michel Laurent, Naperville, IL (US)

Correspondence Address: STEVEN WESEMAN;ASSOCIATE GENERAL COUNSEL, I.P., CABOT MICROELECTRONICS CORPORATION, 870 NORTH COMMONS DRIVE, AURORA, IL, 60504, US

(73) Assignee: Cabot Microelectronics Corporation, Aurora, IL (US)

(21) Appl. No.: 11/153,817

(22) Filed: Jun. 15, 2005

(51) Int. Cl. A61F 2/06; A61K 6/083; A61F 13/00

(52) U.S. Cl. 424/422; 427/2.26; 623/1.11

(57) ABSTRACT

The invention provides a method of preparing a biomedical implant comprising the steps of providing a biomedical implant comprising a metal and having a surface, wherein the surface comprises a metal oxide layer, contacting the biomedical implant with a composition comprising an alkaline earth element, disrupting the metal oxide layer on the surface of the biomedical implant, and adhering the alkaline earth element to the surface of the biomedical implant.

### FIELD OF THE INVENTION

This invention pertains to methods for improving the surfaces of biomedical implants to be surgically implanted into living bone.

### BACKGROUND OF THE INVENTION

The success of a biomedical implant surgically implanted in living bone is dependent upon achieving and maintaining an enduring bond between the confronting surfaces of the biomedical implant and the host bone. For elderly patients, surgeons typically use bone cement to fixate a biomedical implant made of stainless steel alloy or a cobalt-chrome alloy to the host bone. This procedure is inexpensive and usually survives the lifetime of the patient. For younger patients who are more physically active, however, surgeons may use a biomedical implant made of a titanium or tantalum alloy. These alloys are suitable because they are light weight, corrosion-resistant, and flexible. Tantalum-based biomedical implants also have attractive mechanical properties that enable them to be easily fabricated into complex shapes. In place of cement, bone growth around the biomedical implant is desired in order to strengthen the bond between the host bone and biomedical implant.

It is known through clinical experience extending over several decades that titanium and tantalum alloys have the requisite biocompatability with living bone to be acceptable materials for manufacturing biomedical implants when the site of installation is properly prepared to receive them. Methods for preparing living bone to receive a biomedical implant have been known for thirty years or more, but considerable controversy remains concerning the ideal properties for the surface of the biomedical implant which confronts the host bone. Studies have shown that the essential requirement for a biomedical implant to show bioactivity is the formation of a biologically active bonelike apatite layer on the surface of the biomedical implant when placed in the body environment. However, it is also known that in thin films, coatings, and layered materials, surface cracking and debonding, or delamination, are common forms of mechanical failure which must be overcome to produce an enduring bond between the confronting surfaces of the biomedical implant and the host bone.

Prior processes that have been used in attempts to achieve biocompatible surfaces on biomedical implants have taken many forms, including acid etching, ion etching, chemical milling, laser etching, and spark erosion, as well as coating, cladding, and plating, the surface with various materials, for example, bone-compatible apatite materials such as hydroxyapatite, whitlockite, or bone-derived materials.

### BRIEF SUMMARY OF THE INVENTION

The invention provides a method of preparing a biomedical implant comprising the steps of (a) providing a biomedical implant comprising a metal and having a surface, wherein the surface comprises a metal oxide layer, (b) contacting the biomedical implant with a composition comprising an alkaline earth element, (c) disrupting the metal oxide layer on the surface of the biomedical implant, and (d) adhering the alkaline earth element to the surface of the biomedical implant.

### DETAILED DESCRIPTION OF THE INVENTION

The invention provides a method of preparing a biomedical implant. The method comprises the steps of (a) providing a biomedical implant comprising a metal and having a surface, wherein the surface comprises a metal oxide layer, (b) contacting the biomedical implant with a composition comprising an alkaline earth element, (c) disrupting the metal oxide layer on the surface of the biomedical implant, and (d) adhering the alkaline earth element to the surface of the biomedical implant.

The method optionally further comprises the step of forming an apatite layer on a portion of the surface of the biomedical implant.

Alternatively, or in addition, the method optionally further comprises the step of sterilizing the surface of the biomedical implant. Sterilization of the biomedical implant is necessary prior to implantation into a host to prevent infection of the host at the implantation site.

The biomedical implant is any man-made material intended to be inserted into a human body. The biomedical implant can comprise any suitable metal. Suitable metals include, for example, tantalum, titanium, and alloys thereof.

The alkaline earth element can be any suitable alkaline earth element. Suitable alkaline earth elements include, for example, calcium, barium, strontium, and mixtures thereof. In a preferred embodiment, the alkaline earth element is calcium.

The alkaline earth element-containing composition can be any suitable such composition. Typically, the composition will be in liquid or slurry form and will comprise a carrier in addition to the alkaline earth element. The alkaline earth element is present in the composition in any suitable form, but is preferably in the form of an alkaline earth ion. The alkaline earth ions can be provided by any suitable means. For example, the alkaline earth ions of the composition can be provided in the form of an alkaline earth salt.

The alkaline earth element may be present in any suitable concentration in the composition. For example, the alkaline earth element may be present in the composition in a concentration of about 5×10⁻³ to about 7.5 mmoles/kg, about 5×10⁻³ to about 6.5 mmoles/kg, about 5×10⁻³ to about 5 mmoles/kg, about 5×10⁻³ to about 4 mmoles/kg, about 5×10⁻³ to about 3 mmoles/kg, or about 5×10⁻³ to about 2 mmoles/kg.

As indicated above, the composition typically comprises a carrier. Any suitable carrier can be utilized in the composition. The carrier preferably is a liquid carrier, especially a liquid carrier comprising, consisting essentially of, or consisting of water. The water preferably is deionized water. The liquid carrier can further comprise a suitable water-miscible solvent. However, the liquid carrier typically consists essentially of, or entirely of, water, more preferably deionized water.

In addition, the composition can further comprise abrasive particles. The abrasive particles can be any suitable abrasive particles. Suitable abrasives particles include, for example, metal oxide abrasives, such as, alumina (e.g., α-alumina, γ-alumina, δ-alumina, and fumed alumina), ceria, chromia, germania, iron oxide, magnesia, silica (e.g., condensation-polymerized silica, fumed silica, and precipitated silica), titania, zirconia, calcium carbonate, and co-formed products thereof. There are many other suitable abrasives well known in the art, such as, for example, boron carbide, diamond, silicon carbide, titanium nitride, and tungsten carbide. The abrasive can be a mixture of two or more abrasives. In a preferred embodiment, the abrasive particles are silica particles.

The abrasive particles can be of any suitable size, e.g., average particle diamter. Typically, the abrasive particles have an average particle diameter of about 1 μm or less (e.g., about 10-100 nm).

The abrasive particles can be present in the composition in any suitable amount. The total amount of abrasive present in the composition typically is about 0.1 wt. % or more, and preferably about 1 wt. % or more, based on the total weight of the composition. The total amount of abrasive present in the composition typically does not exceed about 25 wt. % (e.g., about 0.1 to 25 wt. %), preferably does not exceed about 20 wt. % (e.g., about 0.1 to 20 wt. %), more preferably does not exceed about 10 wt. % (e.g., about 0.1 to 10 wt. %), and even more preferably does not exceed about 5 wt. % based on the total weight of the composition. In a preferred embodiment, the abrasive particles are present in the composition in an amount of about 1 to about 10 wt. % based on the total weight of the composition.

The composition can comprise an agent that oxidizes the metal. The oxidizing agent can be any suitable oxidizing agent. In a preferred embodiment, the oxidizing agent is hydrogen peroxide.

The oxidizing agent can be present in the composition in any suitable amount. The total amount of oxidizing agent present in the composition typically is about 0.1 wt. % or more, about 0.25 wt. % or more, about 0.50 wt. % or more, about 0.75 wt. % or more, or about 1.0 wt. % or more, based on the total weight of the composition. The total amount of oxidizing agent present in the composition typically does not exceed about 15 wt. % (e.g., about 0.1 to 15 wt. %), about 10 wt. % (e.g., about 0.1 to 10 wt. %), preferably about 8 wt. % (e.g., about 0.1 to 8 wt. %), and more preferably about 5 wt. % (e.g., about 0.1 to 5 wt. %) based on the total weight of the composition. In a preferred embodiment, the oxidizing agent is present in the composition in an amount of about 0.5 to about 8 wt. %, or more preferably in an amount of about 1 to about 5 wt. %, based on the total weight of the composition.

The composition can comprise a surfactant. Suitable surfactants can include, for example, cationic surfactants, anionic surfactants, nonionic surfactants, amphoteric surfactants, mixtures thereof, and the like.

The composition can comprise an antifoaming agent. The antifoaming agent can be any suitable anti-foaming agent. Suitable antifoaming agents include, but are not limited to, silicon-based and acetylenic diol-based antifoaming agents.

The composition can comprise a biocide. The biocide can be any suitable biocide, for example an isothiazolinone biocide.

The composition can have any suitable pH. The pH of the composition can be achieved and/or maintained by any suitable means. More specifically, the composition can further comprise a pH adjustor, a pH buffering agent, or a combination thereof. The pH adjustor can be any suitable pH-adjusting compound. For example, the pH adjustor can be potassium hydroxide, sodium hydroxide, ammonium hydroxide, or a combination thereof. The pH buffering agent can be any suitable buffering agent, for example, phosphates, acetates, borates, sulfonates, carboxylates, ammonium salts, and the like. The composition can comprise any suitable amount of a pH adjustor and/or a pH buffering agent, provided such amount is sufficient to achieve and/or maintain the desired pH of the composition, e.g., within the ranges set forth herein.

It will be appreciated that many of the aforementioned compounds can exist in the form of a salt (e.g., a metal salt, an ammonium salt, or the like), an acid, or as a partial salt. Furthermore, certain compounds or reagents may perform more than one function.

The biomedical implant can be contacted with an alkaline earth element-containing composition in any suitable manner. The step of disrupting the metal oxide layer on the surface of the biomedical implant may be accomplished by any suitable technique. Suitable methods include, for example, subjecting the biomedical implant to sonication in the presence of the composition (e.g., in a bath of the composition), abrading the biomedical implant with the composition, and lapping the surface of the biomedical implant in the presence of the composition. In another embodiment, the step of disrupting the metal oxide layer may be accomplished by spraying a jet of the composition onto the biomedical implant.

The biomedical implant desirably is subjected to chemical-mechanical polishing (CMP) with the composition comprising the alkaline earth element so as to both affect the contact of the biomedical implant with the composition comprising the alkaline earth element and disrupt the metal oxide layer on the surface of the biomedical implant. The CMP of the biomedical implant ideally is accomplished by contacting a surface of biomedical implant with a polishing pad that is moved relative to the biomedical implant with the alkaline earth element-containing composition therebetween, so as to abrade at least a portion of the biomedical implant. In an alternate embodiment, a brush, such as a steel brush, is utilized instead of the polishing pad.

The step of disrupting the metal oxide layer on the surface of the biomedical implant may be accomplished at any suitable temperature. For example, the disruption of the metal oxide layer on the surface of the biomedical implant may be performed at a temperature of about 10° C. to about 40° C. In a preferred embodiment, the disruption of the metal oxide layer on the surface of the biomedical implant may be performed at a temperature of about 20° C. to about 30° C.

The alkaline earth element is adhered to the surface of the biomedical implant by any suitable means. In general, the adherence of the alkaline earth element is achieved merely by the contact of the alkaline earth element with the surface of the biomedical implant. Alternative techniques for adhering the alkaline earth element to the surface of the biomedical implant include ion implantation and physical vapor deposition (both of which may be achieved without disrupting the metal oxide layer on the surface of the biomedical implant). Alternatively, ion implantation can be used to disrupt the metal oxide layer and adhere the alkaline earth element to the surface of the biomedical implant. In one embodiment, the adhesion of the alkaline earth element to the surface of the biomedical implant by physical vapor deposition is performed while simultaneously bombarding the surface of the biomedical implant with high energy ions, desirably in a process known as ion beam assisted deposition. The high energy ions can have any suitable energy, such as an energy of about 10 eV to about 10,000 eV, or from about 20 eV to about 5000 eV, or from about 50 eV to about 500 eV. The high energy ions can be any suitable ions, preferably oxygen ions or inert gas ions, such as, for example, argon ions or neon ions.

The inventive method may comprise other steps. Such other steps may be carried out at any suitable time relative to the other steps. In particular, the other steps may be carried out prior to adhering the alkaline earth element to the surface of the biomedical implant, especially after, but optionally in place of, disrupting the metal oxide layer on the surface of the biomedical implant. Such other steps include: (1) anodizing the biomedical implant in the presence of an alkaline earth ion to form an anodized biomedical implant, or (2) subjecting the biomedical implant to anodic or pulsed electrolysis.

In addition, the biomedical implant may undergo other processing subsequent to the steps of the inventive method. For example, an apatite layer optionally is formed on at least a portion of the surface of the biomedical implant after adherence of the alkaline earth element to the surface of the biomedical implant. Also, the surface of the biomedical implant can be sterilized by any suitable technique at any suitable point in time, preferably subsequent to the aforementioned steps of the inventive method.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed is:

1. A method of preparing a biomedical implant comprising the steps of:
(a) providing a biomedical implant comprising a metal and having a surface, wherein the surface comprises a metal oxide layer,
(b) contacting the biomedical implant with a composition comprising an alkaline earth element,
(c) disrupting the metal oxide layer on the surface of the biomedical implant, and
(d) adhering the alkaline earth element to the surface of the biomedical implant.

2. The method of claim 1, wherein the metal is tantalum, titanium, or an alloy thereof.

3. The method of claim 1, wherein disrupting the metal oxide layer comprises abrading the biomedical implant with the composition.

4. The method of claim 3, wherein abrading the biomedical implant is performed with a polishing pad.

5. The method of claim 1, wherein disrupting the metal oxide layer comprises sonicating the biomedical implant.

6. The method of claim 1, wherein disrupting the metal oxide layer comprises lapping the biomedical implant with the composition.

7. The method of claim 1, wherein disrupting the metal oxide layer comprises spraying a jet of the composition onto the biomedical implant.

8. The method of claim 1, wherein the alkaline earth element comprises at least one alkaline earth element selected from the group consisting of calcium, barium, strontium, and mixtures thereof.

9. The method of claim 8, wherein the alkaline earth element is calcium.

10. The method of claim 1, wherein the composition further comprises:
(i) abrasive particles, and
(ii) a liquid carrier comprising water, and
wherein the composition has a pH of about 7 to about 13.

11. The method of claim 10, wherein the abrasive particles are selected from the group consisting of alumina, ceria, zirconia, calcium carbonate, or silica.

12. The method of claim 11, wherein the abrasive particles are silica particles.

13. The method of claim 10, wherein the composition has a pH of about 8 to about 11.

14. The method of claim 1, wherein the alkaline earth element is present in the composition in a concentration of about 5×10⁻³ to about 7.5 mmoles/kg.

15. The method of claim 10, wherein the abrasive particles are present in the composition in an amount of about 0.1 to about 20 wt. % based on the total weight of the composition.

16. The method of claim 10, wherein the composition further comprises an agent that oxidizes the metal.

17. The method of claim 16, wherein the oxidizing agent is hydrogen peroxide.

18. The method of claim 16, wherein the oxidizing agent is present in the composition in an amount of about 0.5 to about 8 wt. % based on the total weight of the composition.

19. The method of claim 1, wherein the method further comprises:
(e) forming an apatite layer on a portion of the surface of the biomedical implant.

20. The method of claim 1, wherein the method further comprises:
(e) sterilizing the surface of the biomedical implant.

21. The method of claim 1, wherein the metal is tantalum, titanium, or an alloy thereof, and the alkaline earth element comprises at least one alkaline earth element selected from the group consisting of calcium, barium, strontium, and mixtures thereof.

22. A method of preparing a biomedical implant comprising the steps of:
(a) providing a biomedical implant comprising a metal and having a surface, wherein the surface comprises a metal oxide layer,
(b) contacting the biomedical implant with a composition comprising an alkaline earth element,
(c) anodizing the biomedical implant in the presence of the composition to form an anodized biomedical implant, and
(d) adhering the alkaline earth element to the surface of the biomedical implant.

23. The method of claim 22, wherein the alkaline earth element is an alkaline earth ion.

24. The method of claim 22, further comprising:
(e) polishing the anodized biomedical implant.

25. A method of preparing a biomedical implant comprising the steps of:
(a) providing a biomedical implant comprising a metal and having a surface, wherein the surface comprises a metal oxide layer,
(b) subjecting the biomedical implant to anodic or pulsed electrolysis,
(c) contacting the biomedical implant with a composition comprising an alkaline earth element, and
(d) adhering the alkaline earth element to the surface of the biomedical implant.

26. A method of preparing a biomedical implant comprising the steps of:
(a) providing a biomedical implant comprising a metal and having a surface, wherein the surface comprises a metal oxide layer,
(b) providing alkaline earth ions, and
(c) adhering the alkaline earth ions to the surface of the biomedical implant by ion implantation.

27. The method of preparing a biomedical implant comprising the steps of:
(a) providing a biomedical implant comprising a metal and having a surface, wherein the surface comprises a metal oxide layer,
(b) providing an alkaline earth element, and
(c) adhering the alkaline earth element to the surface of the biomedical implant by physical vapor deposition.

28. The method of claim 27, wherein the step of adhering the alkaline earth element to the surface of the biomedical implant is performed while simultaneously bombarding the surface of the biomedical implant with high energy ions.

29. The method of claim 28, wherein the high energy ions are oxygen ions or inert gas ions.

30. The method of claim 28, wherein the high energy ions are argon ions or neon ions.
